(19) United States
(12) Patent Application Publication
(10) Pub. No.: US 2006/0115437 A1
(43) Pub. Date: Jun. 1, 2006

(54) Dental whitening compositions

(76) Inventors: Robert Hayman, Los Angeles, CA (US); Nancy N. Quan, North Hills, CA (US); Jeff MacDonald, Pomona, CA (US)

Correspondence Address: DISCUS DENTAL IMPRESSIONS, INC., 8550 HIGUERA STREET, CULVER CITY, CA, 90232, US

(21) Appl. No.: 11/288,504

(22) Filed: Nov. 28, 2005

Related U.S. Application Data
(63) Continuation-in-part of application No. 11/271,283, filed on Nov. 9, 2005.
(63) Continuation-in-part of application No. 11/271,412, filed on Nov. 9, 2005.
(60) Provisional application No. 60/631,121, filed on Nov. 26, 2004.
(60) Provisional application No. 60/643,309, filed on Jan. 13, 2005.
(60) Provisional application No. 60/653,421, filed on Feb. 15, 2005.
(60) Provisional application No. 60/631,121, filed on Nov. 26, 2004.
(60) Provisional application No. 60/643,309, filed on Jan. 13, 2005.
(60) Provisional application No. 60/653,421, filed on Feb. 15, 2005.

(51) Int. Cl. A61K 8/22

(52) U.S. Cl. 424/53

(57) ABSTRACT

A whitening system has a de-sensitizing effect as well as re-mineralizing capability. The system may be a one-component or a two-component composition. The system may also be an unfoamed, a foamed or a foamable composition. For a one-component system, a sparingly soluble calcium phosphate salt may be in dry particle form with at least one gelling agent, wherein the salt is capable of sustained release of both calcium and phosphate ions into saliva upon exposure of the particle thereto and providing a hydroxyapatite remineralization. In a two component system, a first component has at least one peroxide compound, at least one source of phosphate and at least one gelling agent; and a second component has at least one source of calcium, strontium and/or mixtures thereof. The second component may also contain at least one gelling agent, and the composition maybe present as a foam or is foamable. The composition may be packed in a two-compartment syringe, or be present in a carrier or coated onto a substrate.

### CROSS-REFERENCE TO RELATED APPLICATIONS

This is a continuation-in-part of U.S. patent applications Ser. No. 11/271,283, filed Nov. 9, 2005 entitled “Dental Whitening Systems”, which claims the benefit of U.S. Provisional patent applications Ser. Nos. 60/631,121, filed Nov. 26, 2004, entitled “Whitening System“; 60/643,309, filed Jan. 22, 2005 entitled “Dental Whitening“; and 60/653,421, filed Feb. 15, 2005, entitled “Whitening System Capable of Effective Whitening Action“; and a continuation-in part of U.S. patent application Ser. No. 11/271,412, filed Nov. 9, 2005 entitled “Dental Whitening Compositions”, which claims the benefit of U.S. Provisional patent applications Ser. Nos. 60/631,121, filed Nov. 26, 2004, entitled “Whitening System“; 60/643,309, filed Jan. 22, 2005 entitled “Dental Whitening”; and 60/653,421, filed Feb. 15, 2005, entitled “Whitening System Capable of Effective Whitening Action”; the contents of which are hereby incorporated by reference.

### FIELD OF THE INVENTION

The present invention relates to improvements in tooth treatment compositions. In particular, this invention relates to whitening compositions.

### BACKGROUND OF THE INVENTION

A tooth is comprised of an inner dentin layer and an outer hard enamel that is coated with a protective layer called the acquired pellicle. The enamel layer is composed of hydroxyapatite crystals that create a somewhat porous surface. The pellicle or the enamel can become stained or discolored. It is believed that the porous nature of the enamel layer is what allows staining agents and discoloring substances to permeate the enamel and discolor the tooth.

Many substances that a person's teeth confront or come in contact with on a daily basis can “stain” or reduce the “whiteness” of one's teeth. In particular, food products, tobacco products and fluids such as tea and coffee that one consumes tend to stain one's teeth. These staining and discoloring substances can then permeate the enamel and cause noticeable discoloration of one's teeth. At the same time, some of the food and drinks can cause de-mineralization of the teeth.

One solution to the staining problem is through tooth bleaching. Some dentifrices, like toothpastes, gels, and powders, contain active oxygen or hydrogen peroxide liberating bleaching agents including peroxides, percarbonates and perborates of the alkali and alkaline earth metals or complex compounds containing hydrogen peroxide.

The amount of whitening obtained during tooth bleaching is dependent upon (1) the length of time each day the tray is worn; (2) the number of days the tray is worn; (3) the susceptibility of the teeth to the bleaching agent and (4) the concentration of active peroxides. For maximum whitening, an accelerated treatment time of approximately 18-20 hours per day is recommended.

One concern with some bleaching compositions is that prolonged treatment with highly concentrated bleaching agents present in the composition may contribute to tooth sensitivity following treatment. Even treatments with compositions not known to increase tooth sensitivity in most patients might still cause sensitivity in patients more prone to such sensitivity tendencies.

Another concern is that aggressive brushing, or any treatment with lower pH whitening compositions, as noted in Price et al. (The pH of Tooth-Whitening Products, J Can Dent Assoc, 66:421-6, 2000), may also lead to further de-mineralization of the tooth, decreasing its hardness.

Many attempts have been made to provide dentinal sensitivity relief, sometimes in a separate system from the bleaching system. Other attempts have been made to use a single system to attack both problems. At the same time, attempts have been made to re-mineralize the tooth, again in separate systems.

Therefore, there remains a need for a one-system approach to solving all the above mentioned problems and/or potential problems confronting the tooth.

### SUMMARY OF THE INVENTION

### SUMMARY OF THE INVENTION

The present invention relates to a one-system approach to whitening, sensitivity relief and re-mineralization.

The present invention relates to a whitening composition including at least one source of peroxide, at least one source of calcium and/or strontium, and/or phosphate salt, wherein said salt provides sustained release of calcium and/or strontium and/or phosphorous ions in the presence of water or saliva for forming hydroxyapatite and providing remineralization. The salt may include a calcium or strontium salt of glycerophosphate, lactate, gluconate, fumarate, acetate, chloride, or mixtures thereof; tricalcium phosphate; dicalcium phosphate anhydrous; dicalcium phosphate dehydrate; octacalcium phosphate; tetracalcium phosphate; monocalcium phosphate; or mixtures thereof. The salt is, for example, present in a particulate form having a particle size, for example, of less than about 50 um, and more for example, of from about 1 to about 20 um.

The whitening compositions of the present invention may be present in various forms, including one or two component compositions. In one aspect, the composition may be unfoamed, foamed or foamable. In another aspect, the composition may include a foaming agent. In a further aspect, the composition may be packaged in a container, may be container in a carrier, or may be coated on a substrate.

The foamed, foamable or compositions having a foaming agent may include long lasting foams. In one embodiment, the foamable system may form long lasting, collapsible foams having a half life of at least about 1 hour. In another embodiment, the foamed system may be long lasting, collapsible foams having a half life of at least about a month.

The containers may be used to contain the unfoamed, foamed or foamable compositions. In one embodiment, the container may be a one compartment container. In another embodiment, the container may be a two barrel syringe. In a further embodiment, the container may have separate compartments for the components.

In one aspect, the container or syringe may be provided with a dispensing pump. In another aspect, the container or syringe may be provided with a dispensing tip. In a further aspect, the container or syringe may be provided with a dispensing tip adapted for foaming. In yet a further aspect, the container or syringe may be provided with a dispensing tip including a mixer.

The carrier or substrate may include a substantially insoluble film, a substantially soluble substrate or an erodible film. The carrier or substrate may be adapted for affixing onto at least one tooth by means of an adhesive.

In one embodiment of the invention, the composition may be a one-component composition including at least one source of peroxide, at least one source of soluble or sparingly soluble calcium phosphate salt in particle form, and at least one gelling agent, wherein said salt is capable of sustained release of calcium ions into saliva upon exposure of the particle thereto and providing a hydroxyapatite remineralization upon exposure to a source of phosphate already present in the saliva and/or tooth or teeth. The source of calcium may also include a source of phosphate. The composition may further include at least a separate source of phosphate

In another embodiment, the composition may be a 2-component composition having a first component including at least one source of peroxide, and at least one gelling agent; and a second component including at least one source of soluble or sparingly soluble calcium, strontium and/or mixtures thereof in particulate form and at least one gelling agent, wherein said source of calcium or strontium may provide sustained release of calcium or strontium ions in the presence of water or saliva, for forming hydroxyapatite and providing remineralization upon combination with a source of phosphate already present in the saliva or on the tooth or teeth.

In a further embodiment of the invention, the composition may be a foamed 2-component composition having a first component including at least one source of peroxide, and at least one gelling agent; and a second component including at least one source of calcium, strontium and/or mixtures thereof, wherein said source of calcium may provide sustained release of calcium and/or strontium ions in the presence of water or saliva, for forming hydroxyapatite and providing remineralization upon combination with a source of phosphate already present in the saliva or on the tooth or teeth.

In one embodiment, the first component may also include a de-sensitizing agent. In another embodiment, the second component may also include at least one source of peroxide. In a further embodiment, the first component may also include a source of phosphate. In yet another embodiment, both components may include a de-sensitizing agent. In yet a further embodiment, the first component may also include a gel stabilizer. In still yet another embodiment, the first component may be substantially milky, cloudy, opaque or colored, and the second component may be substantially clear, and vice versa; or both components may be substantially milky, cloudy, opaque or colored. In still yet another embodiment, the two-components of the system are adapted to be admixed and applied to the teeth from a dental tray for sustained contact.

The present invention further relates to a system including a substrate, and coated on at least one side of the substrate is a one-component composition including at least one source of peroxide, and at least one sparingly soluble or soluble calcium and/or strontium salt in dry particle form, wherein said salt is capable of sustained release of both calcium and/or strontium ions into saliva upon exposure of the particle thereto and providing a hydroxyapatite remineralization upon combination with a source of phosphate salt already present in the saliva and/tooth or teeth. The salt may include a calcium or strontium salt of glycerophosphate, lactate, gluconate, fumarate, acetate, chloride or mixtures thereof, wherein said source of calcium and/or strontium may provide sustained release of calcium and/or strontium ions in the presence of water or saliva, for forming hydroxyapatite and providing remineralization upon combination with a source of phosphate already present in the saliva or on the tooth or teeth.

In one embodiment, the sparingly soluble calcium salt may include α-tricalcium phosphate, β-tricalcium phosphate, tetracalcium phosphate, monocalcium phosphate monobasic, dicalcium phosphate anhydrous, dicalcium phosphate dihydrate, octacalcium phosphate or mixtures thereof.

In a further embodiment, in addition to the source of calcium and/or strontium salt, a separate source of phosphate salt in particulate form may be included. These may be sparingly soluble or soluble and may include monosodium phosphate (NaH₂PO₄), disodium phosphate, tetrapotassium pyrophosphate or mixtures thereof.

The present invention still further relates to a system approach for whitening and remineralizing. In one embodiment, the system may include a low peroxide content whitening composition having remineralizing effect. In another embodiment, the system may include a low peroxide content whitening composition having remineralizing effect with sensitivity relief. In yet another embodiment, the system may include a low peroxide content whitening composition having remineralizing effect and fluoride treatment. In a further embodiment, the system may include a low peroxide content whitening composition having remineralizing effect, fluoride treatment and anti-bacterial effect. In others embodiments, any of the combination effects desired may be achieved. In even more embodiments, other agents, including anti-plaque agents, anti-staining agents, vitamin supplements or others that may be beneficial to teeth, breath or even general health care may be included.

Any or all of the systems and compositions disclosed above may be applied to a target for an extended period of at least 60 minutes. The composition or system may be applied directly onto the target or may be present in a tray adapted to surround at least one tooth.

Any or all of the systems and compositions disclosed above may also include a source of at least one peroxide compound, and at least one gelling agent; and a second component including at least one source of calcium, strontium or mixtures thereof, and at least one gelling agent.

The components may be kept apart prior to use in a two-compartment container or syringe as discussed above, or container or coated on a carrier or substrate sequentially to be kept separate until use.

The present invention together with the above and other advantages may best be understood from the following detailed description of the exemplary embodiments of the invention.

### DETAILED DESCRIPTION OF THE INVENTION

The detailed description set forth below is intended as a description of the presently exemplified dental whitening compositions provided in accordance with aspects of the present invention and is not intended to represent the only forms in which the present invention may be prepared or utilized. The description sets forth the features and the steps for preparing and using the dental whitening compositions of the present invention. It is to be understood, however, that the same or equivalent functions and ingredients incorporated in the dental whitening compositions may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein may be used in the practice or testing of the invention, the exemplified methods, devices and materials are now described.

All publications mentioned herein are incorporated herein by reference for the purposes of describing and disclosing, for example, the compositions and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosures by virtue of prior invention.

The whitening composition of the present invention may be a one- or a two-component composition and includes at least one source of peroxide. The one- or two-component composition may be in many different forms, including unfoamed, foamed, foamable or including a foaming agent. Any or all of the mentioned compositions may be packaged in a container, may be contained in a carrier, or may be coated on a substrate.

The source of peroxide may include, for example, metal or metal ion free compounds. Examples of suitable metal ion compounds may include metal percarbonates, perchlorates, peroxy compounds or mixtures thereof. Suitable metal ion free compounds may include hydrogen peroxide and organic peroxides including urea peroxide (carbamide peroxide), glyceryl peroxide, benzoyl peroxide and the like. More for example, peroxides include hydrogen peroxide, carbamide peroxide and mixtures thereof. The total peroxide present in the whitening gel ranges, for example, from about 0.5% by weight to about 45% by weight of the gel, more for example, from about 1% by weight to about 35% by weight of the gel.

When hydrogen peroxide is used, it is usually provided as a 50% aqueous solution. When used alone, the amount of the hydrogen peroxide aqueous solution in the peroxide gel ranges for example, from about 1% to about 60% (0.5% to 30% in the absence of water); more for example, the amount ranges from about 2% to about 40% (1% to 20% in the absence of water).

On the other hand, when carbamide peroxide is used, it is likely to be used in combination with hydrogen peroxide, though it may be used alone.

When used in combination, the carbamide peroxide is generally present, for example, in an amount from about 0% by weight to about 40% by weight, and more for example, in an amount from about 5% to about 35% by weight. At the same time, hydrogen peroxide, for example, is provided as a 50% aqueous solution and is generally present in an amount of from about 0.5% to about 30% (0.25% to 15% in the absence of water); more for example, in an amount of about 1% to about 30% (0.5% to 15% in the absence of water).

The peroxide source may be present in either one or both components of the system in a two-component system. When it is also present in the second component, the combined total source of peroxide content is in the same range as is disclosed above.

For remineralizing effects, soluble or sparingly soluble calcium and/or strontium compounds and/or amorphous calcium and/or strontium compounds, for example, in particulate form may be used. Calcium compounds may include those disclosed in U.S. Pat. Nos. 5,993,786, 5,833,954 and 6,491,900, the contents of all of which are incorporated herein by reference.

The soluble or sparingly soluble calcium and/or strontium salt in particle form is capable of sustained release of calcium and/or strontium ions into saliva or water upon exposure of the particle thereto. The calcium and/or strontium ions may interact with a source of phosphate already present in the saliva and/or tooth to provide a hydroxyapatite remineralization upon prolonged contact with tooth, teeth, of tissue for at least, for example, about one (1) minute, more for example, about five (5) minutes, and even more for example, about ten (10) minutes. This form of a source of calcium and/or strontium compound may be suitable in either a one- or a two-component whitening system.

In one aspect, the soluble or sparing soluble calcium and/or strontium compound may include a calcium and/or strontium salt of glycerophosphate, lactate, gluconate, fumarate, acetate, chloride or mixtures thereof. These may provide sustained release of calcium and/or strontium ions in the presence of water or saliva, for forming hydroxyapatite and providing remineralization upon combination with a source of phosphate already present in the saliva or on the tooth or teeth. In another aspect, a source of phosphate may also be present in addition to the source of calcium or strontium salt, if desired, in the form of a soluble or sparingly soluble salt or compound in particulate form.

In a further aspect, a sparingly soluble calcium phosphate salt in particulate form may be used and may include α-tricalcium phosphate, β-tricalcium phosphate, tetracalcium phosphate, monocalcium phosphate monobasic, dicalcium phosphate anhydrous, dicalcium phosphate dihydrate, octacalcium phosphate or mixtures thereof.

All of sources of calcium and/or strontium salt, calcium phosphate salts, phosphate salt may be provided with a particle size, for example, of less than about 50 um, and more for example, of from about 1 to about 20 um.

For a two-component system, the composition may include a source of a soluble or sparingly soluble calcium and/or strontium compound in particle form that is in a separate component from the component containing the source of peroxide. In one aspect, the soluble or sparing soluble calcium and/or strontium compound may include a calcium and/or strontium salt of glycerophosphate, lactate, gluconate, fumarate, chloride, acetate or mixtures thereof which may provide sustained release of calcium and/or strontium ions in the presence of water or saliva, for forming hydroxyapatite and providing remineralization upon combination with a source of phosphate already present in the saliva or on the tooth or teeth. In another aspect, in addition to the source of calcium and/or strontium salt, if desired, a separate source of a phosphate salt, which may be substantially soluble or a sparingly soluble salt or compound, in particulate from, may be present in the component containing the source of peroxide, and said salts each may be separately combined with a separate gel or paste, which may or may not include a source of peroxide, and wherein said salts are capable, respectively, of sustained release of calcium and/or strontium and phosphorous ions in the presence of water for at least for example, about one (1) minute, more for example, about five (5) minutes, and even more for example, about ten (10) minutes, to form hydroxyapatite as an end product when said gel or paste containing, respectively, said salt and said compound are mixed in the presence of water. The particle size of the source of calcium and/or strontium salt, calcium phosphate, or phosphate salt may be again provided with a particle size, for example, of less than about 50 um, and more for example, of from about 1 to about 20 um.

The compositions of the present invention are particularly suitable for prolonged continuous contact with tooth or teeth, as the longer the contact, the higher the incidences of formation of hydroxyapatite may be realized.

In addition to or in lieu of the sparingly soluble calcium phosphate in particulate form mentioned above, amorphous calcium compounds such as amorphous calcium phosphate (ACP), amorphous calcium phosphate fluoride (ACPF), amorphous calcium carbonate phosphate (ACCP), amorphous calcium carbonate phosphate (ACCP), and amorphous calcium carbonate phosphate fluoride (ACCPF) may also be used in remineralizing teeth. These amorphous compounds are disclosed in U.S. Pat. Nos. 5,037,639, 5,268,167, 5,437,857, 5,562,895, 6,000,341, and 6,056,930, the disclosure of each is hereby incorporated by reference in its entirety.

In addition to amorphous calcium compounds, amorphous strontium compounds such as amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF) and amorphous strontium calcium carbonate phosphate fluoride (ASCCPF) may be used in re-mineralization, as noted above. These compounds are disclosed in U.S. Pat. No. 5,534,244, the content of which is hereby incorporated by reference in its entirety.

Without wishing to be bound to a theory, it is surmised that the soluble or sparingly soluble calcium compounds in particle form described above generally form amorphous compounds on their way to forming hydroxyapatite as an end product. Thus, the soluble or sparingly soluble calcium compounds in particle form may be a precursor of amorphous calcium compounds.

For compositions including both calcium salt in particulate form and amorphous calcium and/or strontium phosphate compounds, it is further surmised that amorphous calcium and/or strontium compounds may be formed quickly upon mixing of the two sources, and upon prolonged contact, more amorphous calcium compounds are additionally formed, all of which generate hydroxyapatite as the end product, therefore providing multiple stages of hydroxyapatite formation.

Some of the compounds mentioned above may also be used in fluoridating teeth. All of the above compounds or solutions which form the amorphous compounds, when applied either onto or into dental tissue, may aid to prevent and/or repair dental weaknesses such as dental caries, exposed roots and dentin sensitivity.

For example, the source of phosphate for forming the amorphous calcium and/or strontium compounds may include monosodium phosphate (NaH₂PO₄), disodium phosphate, tetrapotassium pyrophosphate or mixtures thereof. As discussed above, the second component may include a source of calcium or strontium, and when the two gel components are mixed, may combine with phosphate to form the various amorphous calcium and/or strontium phosphates.

The source of phosphate may be, for example, present in an amount of from about 0.2% to about 5% by weight, more for example, between about 0.2% to about 4% by weight.

The source of calcium, strontium or combinations thereof for the faster formation of amorphous calcium compound may include, for example, a calcium salt, a strontium salt, or mixtures thereof, more for example, a calcium salt such as calcium nitrate, calcium acetate or calcium chloride, in an amount of, for example, from about 0.25% by weight to about 1.5% by weight, more for example, about 0.3% to about 1% by weight.

These concentrations may be useful ranges for either or both amorphous compounds or the sources of calcium and/or strontium salt or phosphate salts in particulate form.

In practice, as much phosphate as possible or practicable may be included. However, amounts of monosodium phosphate in excess of about 4% by weight may tend to affect gel stability.

Surprisingly, the phosphate component present in the composition according to the ranges mentioned above may also act to stabilize the gel. At higher levels, the stabilizing effect gradually disappears.

Additionally, the phosphate salt may further act to adjust the pH of the first component. The pH of the system may be, for example, from about 5 to about 8, more for example, from about 5 to about 6.5, even more for example, from about 5 to about 6.

Surprisingly, the amorphous calcium and/or strontium salts present in the composition may also act as sensitivity relief agents. In fact, the present inventors have found that the de-sensitizing effect provided by amorphous calcium phosphate is at least as effective as, if not more effective than, the typical de-sensitizing agents normally used, including some of those discussed below. Therefore, the presence of amorphous calcium and/or strontium salts may potentially replace traditional de-sensitizing agents.

The foamed, foamable or compositions having a foaming agent may include long lasting foams. In one embodiment, the foamable system may form long lasting, collapsible foams having a half life of at least about 1 hour. In another embodiment, the foamed system may be long lasting, collapsible foams having a half life of at least about a month.

The containers may be used to contain the unfoamed, foamed or foamable compositions. For a one-component system, the source of calcium may be, for example, in a substantially non-aqueous form or dry powder or paste so that any hydroxyapatite is not formed prior to use. The one-component composition may also be contained in a one-compartment container in one embodiment. In another embodiment, the composition may be contained in a carrier or coated onto a substrate, which may be substantially in the form of a film, to form a composite.

In packaging a two component composition of the present invention, any convenient way for effecting the separation of the two components before use may be utilized. In one embodiment, the container may be a two barrel syringe. In a further embodiment, a single container may be compartmentalized so that the two components are housed in separate compartments adapted to be dispensed simultaneously and admixed prior to application on the teeth. Alternatively, the two-component composition may be housed in separate containers from which the respective components are dispensed for admixture just prior to use. In one exemplary embodiment of the present invention, the two components are provided in separate chambers of a dual barrel syringe, such as disclosed in U.S. Pat. Nos. 5,819,988, 6,065,645, 6,394,314, 6,564,972 and 6,698,622, incorporated herein by reference.

In any of the packaging methods described, either one compartment container or syringe or the dual-compartment container or syringe, it may be provided with a dispensing tip, or a dispensing tip including a mixer. The mixers may be dynamic or static. Examples of static mixers may include those also disclosed in U.S. Pat. Nos. 5,819,988, 6,065,645, 6,394,314, 6,564,972 and 6,698,622, incorporated herein by reference. In one aspect, the container or syringe may be provided with a dispensing pump. In another aspect, the container or syringe may be provided with a dispensing tip. In a further aspect, the container or syringe may be provided with a dispensing tip adapted for foaming. In yet a further aspect, the container or syringe may be provided with a dispensing tip including a mixer. Examples of some dynamic mixers may include those disclosed in may include those disclosed in U.S. Pat. Nos. 6,443,612, and 6,457,609; and U.S. Patent Publication No. 2002/0190082; the contents of these are hereby incorporated by reference.

Such dispensing tips or mixers may also be adapted for foaming or the syringe or container is provided with a dispensing pump.

Immediately before use, the two components are mixed together in a 1:2 to a 5:1 ratio (first component to second component in a two-component composition) by actuating the syringe or the container. For example, the gels or foams may be mixed in the 1:1 ratio. The admixed whitening gel or foam is applied to the surface of the teeth directly from the syringe or by means of a dental bleaching tray. Other combinations of the components are contemplated by the present invention, depending on the % variation of ingredients present in each component.

Surprisingly, apart from the components for the amorphous compound, such as the source of phosphate and the source of calcium, strontium or mixture, if the source of phosphate is also present in the composition, the two components may include approximately the same active ingredients. When the two components of the exemplified embodiment are mixed, the two sources may combine to form calcium phosphate. Thus, if only a source of calcium is present in the composition and taking advantage of the source of phosphate already present in the saliva and/or tooth, a one-component composition may be just as effective. When applied to the teeth, the calcium phosphate may precipitate onto the surface of the teeth where it may be incorporated into hydroxyapatite, assisting in remineralization of the tooth enamel, as discussed in U.S. Pat. Nos. 5,037,639, 5,268,167, 5,460,803, 5,534,244, 5,562,895, 6,000,341, 5,993,786, 5,833,954 and 6,491,900, 6,056,930, noted and incorporated by reference above. The activity of the amorphous calcium and/or strontium compounds is not compromised, even if the source of peroxide and other active ingredients are present in the same compartment. This may result in improved manufacturing efficiency.

In addition, to visibly distinguish the two components, if desired, the first component may be made to be substantially milky, cloudy, opaque or colored, while the second component may be made to be substantially clear, or vice versa. In addition, both may be made to be substantially milky, cloudy, opaque or colored, and the distinction is by degree or by color.

Most opacifying agents tend to decrease the stability of peroxide; for example, TiO₂ or ZnO₂ may cause the decomposition of peroxide, making it less effective for its intended purposes. Surprisingly, CaCO₃ is found to be an effective pigment as well as a mixture of mica and TiO₂ stabilized with an EDTA salt. Both were found not to affect the peroxide stability, and a white first component resulted. Other opacifying agents having the same effect is also contemplated.

In another form of packaging for convenient use, the composition may be contained or coated onto a carrier or substrate. The carrier or substrate may include a substantially insoluble film, a substantially soluble substrate or an erodible film. The carrier or substrate may be adapted for affixing onto at least one tooth by means of an adhesive.

The composite may be a multi-layered composite having an adhesive layer and a carrier or film containing a whitening composition and or other active compound in either or both layers. Typically, the composite may be relatively thin, for example, less than about 2 mm thick. The adhesive layer may be on the opposite side of the film or carrier from that of the whitening composition. The adhesive coating enables such carrier or film to be adhesively attached to a desired surface. The film or carrier may be flat, textured, embossed, or contain one or more pockets. The composite may also be substantially planar in shape or generally flat.

Whether a carrier or a film is used, the carrier or film may be substantially water insoluble, substantially water soluble, or water erodible. The film may be cut or fabricated into any desired shape, such as a disc, a square, oval, a rectangle, a parallelepiped, or other shapes that provides convenience for use in application and/or treatment.

The composite may also include a release layer, which may be any material which exhibits less affinity for the adhesive than the adhesive exhibits for itself and for the film carrier. The release layer may include any flexible material such as a layer of plastic film, coated paper, polyethylene, polypropylene, polyester, or other material, and may be releasably affixed to the adhesive coating to protect the adhesive coating prior to application to a desired surface. Such release layers may also be treated, at least on the side adjacent to the adhesive coating with agents such as silicones, fluorocarbons, wax, polyester, or other non-stick tote materials to further enhance ease of separation of the release layer from the adhesive coating.

For a substantially insoluble carrier or film, the carrier or film may include a polymeric film, a sheet of paper, cloth, a woven material, a non-woven material, a foil, a piece of rubber and combinations thereof. The film or carrier may have an

A substantially soluble film or carrier may include those disclosed in U.S. Pat. Nos. 5,800,832 and 6,159,498, the contents of which are incorporated herein by reference. Examples include hydroxyethyl cellulose, hydroxypropyl cellulose, hydroxyethylmethyl cellulose, hydroxypropylmethyl cellulose, polyvinyl alcohol, polyethylene glycol, polyethylene oxide, or ethylene oxide-propylene oxide co-polymers, other water soluble polymers or combinations thereof.

An erodible carrier or film may include those described in U.S. Pat. No. 6,585,997, the content of which is hereby incorporated by reference. Examples may include the soluble polymers or copolymers discussed above, which may be modified to render it water erodible instead of water soluble. For definition purpose, water erodible means a material or substance that does not dissolve in water or bodily fluids in total, but instead disintegrates and completely breaks apart upon exposure to water or bodily fluids. This may be accomplished by coating the soluble film or carrier with a more hydrophobic polymer selected from a group of material such as ethyl cellulose, methyl cellulose, other similar polymers or combinations thereof. The type and amount of hydrophobic polymer used may depend on the time it takes for the composite layer to dissolve. The ratio of the hydrophobic polymer to the water soluble polymer may, for example, range from about 1:2 to about 18:1, and more for example, 1:1 to 10:1.

In addition, the erodible carrier or film may also include plasticizers or other excipients needed to enhance the film forming properties of the polymer. Plasticizing agent may include propylene glycol, polyethylene glycol, or glycerine in a small amount of for example, about 0 to about 2% by weight, to improve the flexibility of the film or carrier layer and to adjust the erosion rate of the composite.

The adhesive layer may include a mucoadhesive which may be selected -from the group consisting of polyacrylic acid, which may or may not be partially crosslinked, sodium carboxymethyl cellulose(NaCMC), polyvinyl pyrrolidone(PVP) or combination thereof. The adhesive layer of the composite may also be water soluble and may also exhibit good instantaneous mucoadhesive properties in the dry, film state. In the case of sodium carboxymethyl cellulose, typical average molecular weights range, for example, between about 50,000 and about 700,000 Daltons, more for example, between about 60,000 and about 500,000 Daltons, with a degree of substitution of for example, about 0.5 and about 1.5, more for example, between about 0.6 and about 0.9. The polyvinyl pyrrolidone may be characterized according to its average molecular weight, for example, between about 5,000 and abut 150,000 Daltons, more for example, between about 10,000 and about 100,000 Daltons. In general, if combinations of, for example, polyvinyl pyrrolidone and polyacrylic acid are used, they may be chosen to form a homogeneous adhesive layer.

For a one-component system, the coating of the whitening composition may be effected by mixing the ingredients in solution, keeping the soluble or sparingly soluble sources of calcium and/or strontium and/or phosphate in particulate form, and applying to the film or carrier using a suitable doctor blade or lab coater apparatus. For a two-component whitening system, the coating may be effected by coating one component at a time, drying, and followed by coating the second component, again, keeping the soluble or sparingly soluble sources of calcium and/or strontium and/or phosphate in particulate form. The coating thickness may, for example, range between about 0 to about 1.5 mm, more for example, between about 0.05 and about 0.4 mm. The amount of solids present in the coating solution, the resulting solution viscosity and coating thickness applied all may determine the amount of the whitening composition to be deposited on the film or carrier. Typically, increasing the hydrophobic polymer to water soluble polymer ratio will provide a composite having a longer residence time, while keeping coating thickness, viscosity, coating solids, polymer composition, and other variables constant. In addition, increasing the coating thickness while keeping all other variables constant may also increase the residence time.

The composite may include other active agents including anti-plaque agents, anti-staining agents, vitamin supplements or others that may be beneficial to teeth, breath or even general health care. These other ingredients may be present in one or both components if two component system is present. In addition, for a two-component system, one of the components may be included in the adhesive composition.

The composition of the present invention may be an unfoamed, foamed or foamable composition. In general, a foamed composition may include the same or higher peroxide concentration as that present in an unfoamed composition. On the other hand, a foamable composition may include a higher peroxide concentration in the unfoamed state so that the concentration of peroxide after foaming may be the same or higher than the level present in a typical gel. For foamable compositions, the amounts of peroxide noted above may represent those in the foamed state.

For an unfoamed gel system, additional components may be added to form a stable gel. These may include gelling agents, gel stabilizers, humectants, and other adjuvants for improving gel consistency, may be added to one or both components.

Gelling agents which may be used in the preparation of whitening gels include, for example, cellulosic gums, fumed silica, for example, CAB-O-SIL fumed silica provided by Cabot Corporation, and emulsifying waxes such as Polawax (emulsifying wax NF) or Crodafos CES (cetearyl alcohol (and) dicetyl phosphate (and) ceteth-10 phosphate), provided by Croda, Inc., and mixtures thereof, in amounts to provide a stable gel. Some examples of cellulosic gum may include ‘Klucel’GF, a hydroxymethyl propylcellulose from Hercules.

In addition to the addition of heat, light and/or chemicals, the amount of whitening obtained during a bleaching process is generally dependent upon (1) the length of time the teeth is in contact with the whitening agent; (2) the number of days the treatment is carried out; (3) the susceptibility of the teeth to the bleaching agent and (4) concentration of active peroxide, as noted above. For maximum whitening, a long treatment time with a highly concentrated bleaching composition is generally recommended, as noted before.

Bleaching activity of a whitening composition is generally dictated by the availability of active peroxides, and not generally by the actual concentration of the sources of peroxide present in the composition. When the source of peroxide is present in a solution, active peroxides are readily available. However, a solution, by its nature, is not easily contained, and/or not amenable for sustain action when applied to a patient's teeth, again because it is difficult to confine it to any desired location for any length of time. Thus, a less concentrated peroxide solution requiring longer contact time to be effective is not practical in a solution environment. A more concentrated solution of peroxide, though more efficient in bleaching, is likewise not suitable in a solution environment because it will not solve the confinement and prolonged contact problem. In addition, any concentrated peroxide solution that may come into contact with soft tissue inside a patient's mouth may potentially cause tissue damage. Therefore, to maintain effective bleaching with good containment so as to minimize potential tissue damage, various gelling agents, thickeners, adhesion promoters and/or similar additives may be used, as discussed above in the unfoamed system. These result in the formation of paste, gels, and similar forms, which are effective whitening systems. It is surmised that some of the additives used, though effective in containment and bleaching, may somewhat decrease the bleaching activity by inhibiting the availability of active peroxides, especially if the composition contain thickeners or adhesion promoters derived from polymers of acrylic acid (carbomer), pyrrolidone analog thickeners, or others. Such additives may lead to diminished whitening capacity of peroxides through ionic and covalent interactions within the gel, and act against the desired effect of tooth whitening.

Foaming may be another way to improve substantivity without some of the above mentioned additives. Foamed compositions may also provide prolonged contact without high amounts of additives that may tend to somewhat inhibit the activity of active peroxides, thus further improving the whitening activity. Foams may be formulated with the same or higher amounts of active ingredients and smaller amounts of inactive ingredients. In a given volume, smaller amounts of inactive ingredients such as gelling agents, thickeners, adhesion promoters or similar may be present in the foamed state compared to unfoamed state. Without wishing to be bound to a theory, it is surmised that air performs the function of an inactive ingredient such as a gelling agent, a thickener, an adhesion promoter or mixtures thereof, to give the needed substantivity; but air molecules in general do not inhibit the availability of active peroxides to the same extent as other inactive ingredients needed for substantivity in an unfoamed gel. Thus, a foamed composition has the substantivity of a composition having higher amounts of ingredients such as gelling agents, thickeners, adhesion promoters or similar inactive ingredients, but with more availability of active peroxides for bleaching action even if the same concentration of peroxide is present. In other words, foaming may in effect substitute air bubbles for gelling agents to create substantivity.

Thus, a foamed or foamable composition of the present invention may increase the rate of whitening activity, if desired, without the problems encountered by solutions. Not only is a foamed composition manageable without confinement problems during use, it may also be capable of delivering a high concentration of active peroxide by not inhibiting the availability of active peroxides, and at the same time, may also seek to fill crevices, sometimes seeping into gaps where gaps are not apparent, thus offering effective whitening action not only to the front surface of a tooth, but surfaces in-between teeth as well. Therefore, foaming may potentially provide all the benefits that are not currently available to other whitening systems.

Foams in general also have lower surface tension than their unfoamed counterparts and may therefore be spread as thin as one molecule thick sheets, while at the same time increasing its surface area, thus covering more area with the same volume of whitening composition than their unfoamed counterparts. The ability to lower surface tension may also make the stains easier to remove.

The foamed bubbles, through the forces of capillary action and lowered surface tension, are also themselves good wetting agents, so that a bleaching solution may penetrate through smaller openings, as noted above. The rapid penetration into the tightest spots may also be aided by the distribution of the sizes and shapes of bubbles. Thus, foams may even cause deeper penetration of active peroxide molecules.

It is further surmised that active peroxides may also be captured inside the bubbles of foams. As the bubbles collapse, the active peroxide agent is released to perform whitening actions.

The foamed or foamable composition of the present invention not only has advantages over unfoamed gels, as discussed above, such as flowing between enamel rods, and insuring that more active ingredients get to the areas not previously reachable, but may also provide better sustained action in a home use setting.

For prolonged or for home use, a tray is generally used for sustain contact between the whitening composition and the surface of tooth or teeth to effect whitening. Compositions in the art generally use additives having high adhesion strength to help retain the composition and even the tray in place. Foams may provide such sustain contact without resorting to the use of the kind of adhesion promoting additives used in the art. Foams may also more evenly spread any stress exerted on the teeth or tray. In addition, foamed compositions have lower density and thus are more amenable for sustain contact without gravity drag. Thus, foams not only facilitate the whitening action but may also facilitate the ease of application, penetration into difficult to reach areas, retention and removal. It is further surmised that the active whitening ingredients may be captured inside the bubbles and such bubbles may serve as controlled whitening devices by metering the amount of active peroxides acting on the teeth over time as the bubbles collapse.

In some embodiments, the foamed composition may be made during manufacturing of the gels and may be packaged accordingly. Foam formation may be effected by any agitation or whipping action, use of foaming agents and combinations thereof. Such foams may be made to be long lasting with a desired shelf life, for example, at least about a month.

In other embodiments, foaming agents may be present to render the composition foamable so that foams are generated just prior to use. Some foaming agents may also aid in increasing the adhesion of the gel to a solid surface by allowing it to spread over a greater surface area.

In still other embodiments, foams may be formed just prior to use by agitation with or without any foaming agent.

Thus, some compositions include at least one foaming agent. Different categories of foaming agents are suitable, and they may produce foams in different ways. Suitable foaming agents may include certain surfactants such as anionic, nonionic, amphoteric, zwitterionic, cationic, or mixtures thereof.

Some of these aids in foam formation and some do not. Some surfactants are useful purely for their foaming properties alone, some act only as emulsifiers or wetting agents without foaming, and some even act to reduce foaming. The functions of the surfactants are not necessarily determined by the category they belong to.

Anionic surfactants include, but not limited to water-soluble salts of alkyl sulfates having from about 8 to about 20 carbon atoms in the alkyl radical (such as sodium alkyl sulfate), water-soluble salts of sulfonated monoglycerides of fatty acids having from about 8 to about 20 carbon atoms and mixtures thereof. Examples of anionic surfactants include Sodium lauryl sulfate, sodium coconut monoglyceride sulfonates, phospholipids, sarcosinates, such as sodium lauryl sarcosinate, taurates, sodium lauryl sulfoacetate, sodium lauroyl isethionate, sodium laureth carboxylate, and sodium dodecyl benzenesulfonate. Many of these anionic surfactants are disclosed in U.S. Pat. No. 3,959,458, the content of which is incorporated herein in its entirety by reference.

Nonionic surfactants may include, but not limited, to compounds including a hydrophilic and hydrophobic components (which maybe produced by the condensation of alkylene oxide groups (hydrophilic in nature) with an organic hydrophobic compound which may be aliphatic or alkyl-aromatic in nature). Examples of suitable nonionic surfactants include low viscosity poloxamers (sold under the trade name Pluronic), low viscosity hydroxyethyl cellulose, polysorbates, polyoxyethylene sorbitan esters (sold under the trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures thereof.

Amphoteric surfactants may include, but is not limited to derivatives of aliphatic secondary and tertiary amines in which the aliphatic component may be a straight chain or branched and one of the aliphatic substituents contains from about 8 to about 18 carbon atoms and one contains an anionic water-solubilizing group, such as carboxylate, sulfonate, sulfate, phosphate, phosphonate, betaines, specifically cocamidopropyl betaine, and mixtures thereof.

Many of these nonionic and amphoteric surfactants are disclosed in U.S. Pat. No. 4,051,234, the content of which is incorporated herein by reference in its entirety.

In the present invention, the exemplified surfactants, when used in foamable compositions, are those that not only have some foaming capabilities, but also those with some ability to act as wetting agents.

Actually, any asymmetrical molecule dissolved in water will make at least a weak surfactant. Such weak surfactants may normally not be an effective foaming agent, but its effectiveness may be improved if a foaming dispenser is used. Asymmetrical molecules as used herein include those that include a hydrophilic and a hydrophobic segment, such as some of the nonionic surfactants mentioned above. One end of the molecule is thus polar in nature and dissolves in water, while the other end is nonpolar in nature and avoids water. When in water, the surfactant molecules oriented themselves with their polar ends towards the water molecules, leaving the nonpolar ends free to attract nonpolar molecules. It is surmised that in a foamed or foamable composition of the present invention, the non-polar ends help to lift tooth surface stains, allowing them to be washed away with the water.

The amount of foaming agents may range, for example, from about 0.1% to about 5% by weight of the foamable composition, more for example, from about 0.5% to about 3% by weight, even more for example, less than about 1% by weight.

Still other foaming agents may include reaction products of any base with an acid. These may include, for example, an alkali metal carbonate or bicarbonate, such as sodium bicarbonate, potassium bicarbonate, sodium carbonate or potassium-carbonate, or an alkaline metal carbonate or bicarbonate such as magnesium or calcium bicarbonate or carbonate. The amount used may range, for example, from about 1% to about 10% by weight, more for example, from about 3% to about 7%, still more for example, from about 3.5 to about 5.5% by weight of the composition. The amount used may also depend on the volume of foam required. Thus, by varying the amount of foaming agents, the amount of foam produced may be varied accordingly.

Generally, the ratio of acid and base ranges, for example, from about 1:0.5 to 1:25, more for example, from about 1:1 to 1:4, by weight. Suitable acids include strong acids such as water soluble carboxylic acids, phosphoric acids, nitric acids, and/or sulfuric acids.

Additionally, a foaming agent may also be a gaseous material. The gaseous material may be any inert gas or a gas generated by mixing a basic peroxide solution with an acid solution.

Since the whitening activity results when active peroxide comes into contact with the tooth, the foams generated just prior to use may for example, not need to be too long lasting, if the active ingredients are surmized to be captured within the bubbles, but have a substantive body so that the amount of liquid formed, i.e., the collapsing of the foam bubbles to release active peroxide, may be controlled and balanced. Therefore, the exemplary compositions are not only foamable, but are also capable of producing longer lasting, collapsible foams.

The foams generated by a foamable composition generally have half lives of, for example, from about 1 to 10 hours, more for example, from about 2 to 5 hours. A foam having a half life of 2 hours means that 50% of the bubbles would collapse to release the encapsulated whitening agent, or that the volume of the foam is reduced by 50% in about 2 hours after formation, and 75% of all the bubbles are gone, or the volume of the foam is reduced by 75% in about 4 hours.

The collapse time or half lives of the foam depends on a number of factors. For example, the greater the amount of bubbles formed, the longer the collapse time. Also, the lower the viscosity of the thickeners, and/or other inactive ingredients, the shorter the collapse time. The collapse time may also depend on the nature of the other additives to the, composition, which have surface active properties e.g., surfactants or preservatives as well as the environment. For example, a drier environment may contribute to faster collapse of bubbles.

For foamed compositions, the collapsible bubbles typically have very extended half lives when confined in the package, for example, at least about a month, more for example, at least about three months. During use, the collapse of the foamed bubbles is aided by the environment, such as the loss of water or solvent to the environment, and the saliva in the patient's mouth. A packaging may be designed so that a foamed composition may have a desired shelf life after the package or container has been opened and the remaining has been exposed to air. A single-use packaging may also be designed.

For example, a lower viscosity gelling agent or thickener may be used. They are not as likely to inhibit the availability of active peroxides to the same extent as a higher viscosity gelling agent. The viscosity is for example, generally less than about 10,000 cps, more for example, less than about 8,000 cps, and even more for example, less than about 5,000 cps.

Suitable foaming agents include foamable surfactants including at least some sodium lauryl Sulfate as the primary foaming agent.

As discussed above, substantivity, i.e. the ability of a product to linger, is a desirable property in any whitening composition. On the other hand when the desired property of a product is the ability to be rinsed off easily, a foaming surfactant would not be used. However, there is a general desire that a whitening composition may both have substantivity and the ease to be rinsed off. When this is desired, foams generated in a “foaming pump”, a dispensing tip adapted for foaming, or a dispensing tip including a mixer adapted for foaming may be used. Such foaming devices again may produce foams with desirable properties, while using a minimum of amount of surfactants, for example, less than about 0.5%. At the same time, some combinations of additives may be chosen to produce the desired effect as well, such as by the addition of other foaming agents.

Additional de-sensitizing agents may also be used. Suitable desensitizing agents, if added, may include, for example, alkali nitrates such as potassium nitrate, sodium nitrate and lithium nitrate; and other potassium salts such as potassium chloride and potassium bicarbonate.

The percent of desensitizing agent may be present, for example, up to about 5 percent by weight, more for example, up to about 4 percent by weight, and even more for example, up to about 3 percent by weight.

The de-sensitizing agent may be present in both components of the system and the ranges present above may represent the total in both components. For example, the de-sensitizing agent may be present in approximately equal amounts in each component.

Surprisingly, the de-sensitizing agent also acts to stabilize the gel.

In addition, optional additives including humetants, flavorings, coloring agents, anti-plaque agents, anti-staining compounds, pH adjusting agents, excipients such as emollients, preservatives, other types of stabilizers such as antioxidants, chelating agents, tonicity modifiers (such as sodium chloride, manitol, sorbitol or glucose), spreading agents, and water soluble lubricants, such as propylene glycol, glycerol or polyethylene glycol. The concentration of each may easily be determined by a person skilled in the art. Some of these may be contemplated for a foamed or foamable system.

The humectants contemplated for use in the inventive compositions include water, polyols, such as glycerol, sorbitol, polyethylene glycols, propylene glycols, hydrogenated partially hydrolyzed polysaccharides and the like. A single humectant or a combination is also contemplated. They are generally present in amounts of, for example, up to about 85%, more for example, from about 15% to about 75% of the formulation. For foamable compositions, the amount of humectant present tends more towards the high end of the range.

As mentioned above, lower peroxide content compositions may be contemplated. These low peroxide compositions may be used at home over longer period of time and may be used as a vehicle for the delivery of many other agents that may be beneficial to oral and general health. These compositions may also be used after an in-office whitening treatment to maintain the effect. In addition, a remineralizing composition adapted for prolong use may be beneficial to damaged tooth or teeth, severe caries, or even bone loss.

In one embodiment, the system may include a low peroxide content whitening composition having remineralizing effect. In another embodiment, the system includes a low peroxide content whitening composition having remineralizing effect with sensitivity relief. In yet another embodiment, the system may include a low peroxide content whitening composition having remineralizing effect and fluoride treatment. In a further embodiment, the system may include a low peroxide content whitening composition having remineralizing effect, fluoride treatment and anti-bacterial effect.

In more embodiments, any of the combination effects desired may be achieved. In even more embodiments, other agents, including anti-plaque agents, anti-staining agents, vitamin supplements or others that may be beneficial to teeth, breath or even general health care may be included.

The peroxide content may be in the low end of the range mentioned above, for example, from about 0.5 to about 5%, more for example, from about 0.5 to about 3%. The composition may be foamed or unfoamed.

Though the amorphous calcium and/or strontium salts present in the composition may also act as sensitivity relief agents, additional de-sensitizing agents, such as the potassium salts and similar mentioned above may also be added for additional effect.

Useful fluoridating agents may include metal fluoride salts such as sodium fluoride, sodium monofluorophosphate, potassium fluoride, lithium fluoride, ammonium fluoride, stannous fluoride; zinc ammonium fluoride, tin ammonium fluoride, calcium fluoride and cobalt ammonium fluoride, and water soluble amine hydrofluorides. Generally, sodium fluoride and stannous fluoride.

Some amorphous calcium and strontium salts may also be useful fluoridation agents and include amorphous calcium phosphate fluoride (ACPF), amorphous calcium carbonate phosphate fluoride (ACCPF) (as disclosed in U.S. Pat. Nos. 5,037,639, 5,268,167, 5,437,857, 5,562,895, 6,000,341, and 6,056,930, the disclosure of each is hereby incorporated by reference in its entirety), amorphous strontium phosphate fluoride (ASPF), amorphous strontium carbonate phosphate fluoride (ASCPF) and amorphous strontium calcium carbonate phosphate fluoride (ASCCPF) (as disclosed in U.S. Pat. No. 5,534,244, the content of which is hereby incorporated by reference in its entirety). These compounds may have both remineralizing and fluoridating effects.

Useful antibacterial agents include, for example, phenolics and salicylamides, and sources of certain metal ions such as zinc, copper, silver and stannous ions, for example in salt form such as zinc, copper and stannous chloride, and silver nitrate. These are again present in small quantities when used.

In foamed or foamable form, the advantages of foams mentioned above are also realized in these low peroxide content whitening compositions.

The present invention is further described by the following examples. The ingredients in B were mixed in a Kitchenaide mixer until the hydroxymethyl propylcelluose, available from Hercules Incorporated, Aqualon Division, Hercules Plaza (Wilmington, Del.) and the emulsifying wax were dispersed in the propylene glycol. Then, the ingredients in A were added and mixed together prior to the addition of the ingredients in C. Afterwards, the flavoring and silica in C were added to the thickened dispersion with moderate mixing until dispersed. Finally, if used, Timica Extralarge Sparkle 110S (titanium dioxide/mica, available from Engelhard Corporation, Iselin, N.J.) was added and mixed under vacuum to remove all air/foam. On the other hand, when foamed compositions are made, such foam removal action is not needed, while at the same time, agitation for additional foam production is used. Also, for foamable compositions, foaming agents are added. In either foamed or foamable compositions, a decreased amount of gelling agents, thickeners and/or other inactive ingredients may or may not be present.

First Component, Composition 1

| | | % | Grams | Phase |
|---|---|---|---|---|
| 50% Hydrogen peroxide | 50% water | 3.62 | 10.86 | A |
| TKPP | tetrapotassium pyrophosphate | 0.2 | 0.6 | A |
| Glycerin | | 20 | 60 | A2 |
| carbamide peroxide | urea hygrogen peroxide | 17 | 51 | A2 |
| Antifoaming Agent | silicone emulsion/Dimethyl polysiloxan | QS | QS | A2 |
| | emulsion/“Dow” | | | |
| Klucel GF | hydrxypropyl methyl cellulose “Hercules” | 1.981 | 105 | B |
| propylene glycol | “Westco” | 45.733.019 | | |
| Propylene Glycol | “Westco” | 12.68 | 38.04 | B |
| Polawax NF | emulsifying wax NF “Croda” | 6.5 | 19.5 | B |
| Combine A & B in | | | | C |
| “Kitchenaide” | | | | |
| Cabosil EH-5 | fumed silica/“Cabot” | 4.5 | 13.5 | C |
| Natural Mint | natural mint flavoring | 0.5 | 1.5 | C |

First Component, Composition 2

| | % | Grams | Phase |
|---|---|---|---|
| Water | 4.62 | 13.86 | A |
| TKPP | 0.2 | 0.6 | A |
| Glycerin | 20 | 60 | A2 |
| carbamide peroxide | 16 | 48 | A2 |
| Antifoaming agent | QS | QS | A2 |
| Klucel GF | 35 | 105 | B |
| Propylene Glycol | 12.68 | 38.04 | B |
| Polawax NF | 6.5 | 19.5 | B |
| Combine A & B in “Kitchenaide” | | | C |
| Cabosil EH-5 | 4.5 | 13.5 | C |
| Mint | 0.5 | 1.5 | C |
| | 100 | | |

First Component, Composition 3

| | % | Grams | Phase |
|---|---|---|---|
| Water | 6.82 | 20.46 | A |
| 50% H₂0₂ | 5.8 | 17.4 | A |
| TKPP | 0.2 | 0.6 | A |
| Glycerin | 20 | 60 | A2 |
| carbamide peroxide | 8 | 24 | A2 |
| Antifoaming agent | QS | QS | A2 |
| Klucel GF | 35 | 105 | B |
| Propylene Glycol | 12.68 | 38.04 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | | |

First Component, Composition 4

| | % | Grams | Phase |
|---|---|---|---|
| Water | 9 | 27 | A |
| 50% H₂0₂ | 5.8 | 17.4 | A |
| TKPP | 0.2 | 0.6 | A |
| Glycerin | 19 | 57 | A2 |
| carbamide peroxide | 8 | 24 | A2 |
| Antifoaming agent | QS | QS | A2 |
| Klucel GF | 34.5 | 103.5 | B |
| Propylene Glycol | 12 | 36 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | | |

First Component, Composition 5

| | % | Grams | Phase |
|---|---|---|---|
| Water | 12.5 | 37.5 | A |
| 50% H₂0₂ | 4.2 | 12.6 | A |
| TKPP | 0.2 | 0.6 | A |
| Glycerin | 16.8 | 50.4 | A2 |
| carbamide peroxide | 6 | 18 | A2 |
| Antifoaming agent | QS | QS | A2 |
| Klucel GF | 37 | 111 | B |
| Propylene Glycol | 11.8 | 35.4 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | | |

First Component, Composition 6

| | % | Grams | Phase |
|---|---|---|---|
| Water | 12.5 | 37.5 | A |
| 50% H₂0₂ | 4.2 | 12.6 | A |
| TKPP | 0.2 | 0.6 | A |
| KNO₃ | 2 | 6 | |
| Glycerin | 16.3 | 48.9 | A2 |
| carbamide peroxide | 6 | 18 | A2 |
| Antifoaming agent | QS | QS | A2 |
| Klucel GF | 37 | 111 | B |
| Propylene Glycol | 10.55 | 31.65 | B |
| Polawax NF | 6 | 18 | B |
| Combine A & B in“Kitchenaide” | | | C |
| Timica blend | 0.25 | 0.75 | |
| Cabosil EH-5 | 4.5 | 13.5 | C |
| Natural Mint | 0.5 | 1.5 | C |
| | 100 | 300 | |

First Component, Composition 7

| | % | Grams | Phase |
|---|---|---|---|
| Water | 12.5 | 37.5 | A |
| 50% H₂0₂ | 4.2 | 12.6 | A |
| TKPP | 0.2 | 0.6 | A |
| KNO₃ | 2 | 6 | |
| Glycerin | 16.3 | 48.9 | A2 |
| carbamide peroxide | 6 | 18 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 8

| | % | Grams | Phase |
|---|---|---|---|
| Water | 12.5 | 38.1 | A |
| 50% H₂0₂ | 4.2 | 12.6 | A |
| TKPP | 0.2 | 0.6 | A |
| Glycerin | 16.8 | 50.4 | A2 |
| carbamide peroxide | 6 | 18 | A2 |
| Antifoaming agent | QS | QS | A2 |
| Klucel GF | 37 | 111 | B |
| Propylene Glycol | 11.8 | 35.4 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | | |

First Component, Composition 9

| | % | Grams | Phase |
|---|---|---|---|
| Water | 12.5 | 37.5 | A |
| 50% H₂0₂ | 4.2 | 12.6 | A |
| TKPP | 0.2 | 0.6 | A |
| Na₂HPO₄ (di-sodium) | 0.5 | 1.5 | A |
| NaH₂PO₄ (monosodium) | QS | QS | A |
| KNO₃ | 2 | 6 | A |
| Glycerin | 16 | 48 | A2 |
| carbamide peroxide | 6 | 18 | A2 |
| Antifoaming agent | QS | QS | A2 |
| Klucel GF | 37 | 111 | B |
| Propylene Glycol | 9.95 | 29.85 | B |
| Polawax NF | 6 | 18 | B |
| Combine A & B in “Kitchenaide” | | | C |
| Timica blend/Titanium Dioxide and Mica/ | 0.5 | 1.5 | |
| “Englehard” | | | |
| TiO₂ | 0.25 | 0.75 | |
| Cabosil EH-5 | 4.4 | 13.2 | C |
| Natural Mint | 0.5 | 1.5 | C |
| | 100 | 300 | |

First Component, Composition 10

| | % | Grams | Phase |
|---|---|---|---|
| Water | 12.7 | 38.1 | A |
| 50% H₂0₂ | 0 | 0 | A |
| TKPP | 0.2 | 0.6 | A |
| Na₂HPO₄ (di-sodium) | 0.5 | 1.5 | A |
| NaP₂PO₄ (monosodium) | QS | QS | A |
| KNO₃ | 2 | 6 | A |
| Glycerin | 14 | 42 | A2 |
| carbamide peroxide | 12 | 36 | A2 |
| Antifoaming agent | QS | QS | A2 |
| Klucel GF | 37 | 111 | B |
| Propylene Glycol | 9.95 | 29.85 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.4 | 13.2 | C |
| Natural Mint | 0.5 | 1.5 | C |
| | 100 | 300 | |

First Component, Composition 11

| | % | Grams | Phase |
|---|---|---|---|
| Water | 12.5 | 37.5 | A |
| 50% H₂0₂ | 4.2 | 12.6 | A |
| TKPP | 0.2 | 0.6 | A |
| NaH₂PO₄ (monosodium) | 0.5 | 1.5 | A |
| Na₂HPO₄ (di-sodium) | QS | QS | A |
| KNO₃ | 2 | 6 | A |
| Glycerin | 16 | 48 | A2 |
| carbamide peroxide | 6 | 18 | A2 |
| Antifoaming agent | QS | QS | A2 |
| Klucel GF | 37 | 111 | B |
| Propylene Glycol | 9.95 | 29.85 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.4 | 13.2 | C |
| Natural Mint | 0.5 | 1.5 | C |
| | 100 | 300 | |

First Component, Composition 12

| | % | Grams | Phase |
|---|---|---|---|
| Water | 8.3 | 24.9 | A |
| 50% H₂0₂ | 8.4 | 25.2 | A |
| TKPP | 0.2 | 0.6 | A |
| KNO₃ | 2 | 6 | |
| Glycerin | 16.3 | 48.9 | A2 |
| carbamide peroxide | 6 | 18 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 13

| | % | Grams | Phase |
|---|---|---|---|
| Water | 12 | 37.5 | A |
| 50% H₂0₂ | 4.2 | 12.6 | A |
| TKPP | 0.2 | 0.6 | A |
| KNO₃ | 2 | 6 | |
| Glycerin | 16.3 | 48.9 | A2 |
| carbamide peroxide | 11.5 | 34.5 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 316.5 | |

First Component, Composition 14

| | % | Grams | Phase |
|---|---|---|---|
| Water | 12.3 | 36.9 | A |
| 50% H₂0₂ | 4.2 | 12.6 | A |
| TKPP | 0.2 | 0.6 | A |
| KNO₃ | 2 | 6 | |
| Glycerin | 11 | 33 | A2 |
| carbamide peroxide | 11.5 | 34.5 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 15

| | % | Grams | Phase |
|---|---|---|---|
| Water | 9.5 | 28.5 | A |
| 50% H₂0₂ | 10.1 | 30.3 | A |
| TKPP | 0.2 | 0.6 | A |
| KNO₃ | 2 | 6 | |
| Glycerin | 11.3 | 33.9 | A2 |
| carbamide peroxide | 8.1 | 24.3 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 16

| | % | Grams | Phase |
|---|---|---|---|
| Water | 12.5 | 37.5 | A |
| 50% H₂0₂ | 5.3 | 15.9 | A |
| TKPP | 0.2 | 0.6 | A |
| KNO₃ | 2 | 6 | |
| Glycerin | 9.65 | 28.95 | A2 |
| carbamide peroxide | 14.6 | 43.8 | A2 |
| Antifoaming agent | QS | QS | A2 |
| Klucel GF | 37 | 111 | B |
| Propylene Glycol | 7 | 21 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 17

| | % | Grams | Phase |
|---|---|---|---|
| Water | 5 | 15 | A |
| 50% H₂0₂ | 11.8 | 35.4 | A |
| TKPP | 0.2 | 0.6 | A |
| KNO₃ | 2 | 6 | |
| Glycerin | 6 | 18 | A2 |
| carbamide peroxide | 16.2 | 48.6 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 18

| | % | Grams | Phase |
|---|---|---|---|
| Water | 9.2 | 27.6 | A |
| 50% H₂0₂ | 7.7 | 23.1 | A |
| TKPP | 0.2 | 0.6 | A |
| KNO₃ | 2 | 6 | A |
| Glycerin | 16.6 | 49.8 | A2 |
| carbamide peroxide | 5.5 | 16.5 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | C |
| TiO₂ | 0.25 | 0.75 | C |
| Cabosil EH-5 | 4.5 | 13.5 | C |
| Natural Mint | 0.5 | 1.5 | C |
| | 100 | 300 | |

First Component, Composition 19

| | % | Grams | Phase |
|---|---|---|---|
| Water | 9.2 | 27.6 | A |
| 50% H₂0₂ | 7.7 | 23.1 | A |
| TKPP | 0.2 | 0.6 | A |
| KNO₃ | 2 | 6 | A |
| Glycerin | 16.6 | 49.8 | A2 |
| carbamide peroxide | 5.5 | 16.5 | A2 |
| Antifoaming agent | QS | QS | A2 |
| Klucel GF | 37 | 111 | B |
| Propylene Glycol | 10.05 | 30.15 | B |
| Crodaphos | 6 | 18 | B |
| Combine A & B in “Kitchenaide” | | | C |
| Timica blend | 0.5 | 1.5 | C |
| TiO₂ | 0.25 | 0.75 | C |
| Cabosil EH-5 | 4.5 | 13.5 | C |
| Natural Mint | 0.5 | 1.5 | C |
| | 100 | 300 | |

First Component, Composition 20

| | % | Grams | Phase |
|---|---|---|---|
| Water | 9.5 | 28.5 | A |
| 50% H₂0₂ | 10.1 | 30.3 | A |
| TKPP | 0.2 | 0.6 | A |
| KNO₃ | 2 | 6 | |
| Glycerin | 11.3 | 33.9 | A2 |
| carbamide peroxide | 8.1 | 24.3 | A2 |
| Antifoaming agent | QS | QS | A2 |
| Klucel GF | 37 | 111 | B |
| Propylene Glycol | 10.05 | 30.15 | B |
| Crodaphos | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 21

| | % | Grams | Phase |
|---|---|---|---|
| Water | 8.3 | 24.9 | A |
| 50% H₂0₂ | 8.4 | 25.2 | A |
| TKPP | 0.2 | 0.6 | A |
| KNO₃ | 2 | 6 | |
| Glycerin | 17.8 | 53.4 | A2 |
| carbamide peroxide | 4.5 | 13.5 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 22

| | % | Grams | Phase |
|---|---|---|---|
| Water | 8.3 | 24.9 | A |
| 50% H₂0₂ | 7.3 | 21.9 | A |
| TKPP | 0.2 | 0.6 | A |
| KNO₃ | 2 | 6 | |
| Glycerin | 17.3 | 51.9 | A2 |
| carbamide peroxide | 6.1 | 18.3 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 23

| | % | Grams | Phase |
|---|---|---|---|
| Water | 8.3 | 24.9 | A |
| 50% H₂0₂ | 7.3 | 21.9 | A |
| TKPP | 0.2 | 0.6 | A |
| KNO₃ | 2 | 6 | |
| Glycerin | 17.3 | 51.9 | A2 |
| carbamide peroxide | 6.1 | 18.3 | A2 |
| Antifoaming agent | QS | QS | A2 |
| Klucel GF | 37 | 111 | B |
| Propylene Glycol | 10.55 | 31.65 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0 | 0 | C |
| | 100 | 300 | |

First Component, Composition 24

| | % | Grams | Phase |
|---|---|---|---|
| Water | 8.3 | 41.5 | A |
| 50% H₂0₂ | 8.4 | 42 | A |
| TKPP | 0.2 | 1 | A |
| KNO₃ | 2 | 10 | |
| Glycerin | 18.3 | 91.5 | A2 |
| carbamide peroxide | 4.5 | 22.5 | A2 |
| Antifoaming agent | QS | QS | A2 |
| Klucel GF | 37 | 185 | B |
| Propylene Glycol | 10.05 | 50.25 | B |
| Polawax NF | 6 | 30 | B |
| Combine A & B in “Kitchenaide” | | 0 | C |
| Timica blend | 0.5 | 2.5 | |
| TiO₂ | 0.25 | 1.25 | |
| Cabosil EH-5 | 4.5 | 22.5 | C |
| Natural Mint | 0 | 0 | C |
| | 100 | 500 | |

First Component, Composition 25

| | % | Grams | Phase |
|---|---|---|---|
| Water | 12.5 | 37.5 | A |
| 50% H₂0₂ | 3.3 | 9.9 | A |
| TKPP | 0.2 | 0.6 | A |
| KNO₃ | 2 | 6 | |
| Glycerin | 17.7 | 53.1 | A2 |
| carbamide peroxide | 5.5 | 16.5 | A2 |
| Antifoaming agent | QS | QS | A2 |
| Klucel GF | 37 | 111 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 26

| | % | Grams | Phase |
|---|---|---|---|
| Water | 12.5 | 37.5 | A |
| 50% H₂0₂ | 3.3 | 9.9 | A |
| TKPP | 0.2 | 0.6 | A |
| KNO₃ | 0 | 0 | |
| Glycerin | 18.7 | 56.1 | A2 |
| carbamide peroxide | 5.5 | 16.5 | A2 |
| Antifoaming agent | QS | QS | A2 |
| Klucel GF | 38 | 114 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 27

| | % | Grams | Phase |
|---|---|---|---|
| Water | 8.3 | 24.9 | A |
| 50% H₂0₂ | 8.4 | 25.2 | A |
| TKPP | 0.2 | 0.6 | A |
| KNO₃ | 0 | 0 | |
| Glycerin | 18.8 | 56.4 | A2 |
| carbamide peroxide | 4.5 | 13.5 | A2 |
| Antifoaming agent | QS | QS | A2 |
| Klucel GF | 38 | 114 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 28

| | % | Grams | Phase |
|---|---|---|---|
| Water | 9.5 | 28.5 | A |
| 50% H₂0₂ | 10.1 | 30.3 | A |
| TKPP | 0.2 | 0.6 | A |
| KNO₃ | 0 | 0 | |
| Glycerin | 12.3 | 36.9 | A2 |
| carbamide peroxide | 8.1 | 24.3 | A2 |
| Antifoaming agent | QS | QS | A2 |
| Klucel GF | 38 | 114 | B |
| Propylene Glycol | 10.05 | 30.15 | B |
| Polawax NF | 6 | 18 | B |
| Combine A & B in “Kitchenaide” | | | C |
| Timica blend | 0.5 | 1.5 | |
| TiO₂ | 0.25 | 0.75 | |
| Cabosil EH-5 | 4.5 | 13.5 | C |
| Natural Mint | 0.5 | 1.5 | C |
| | 100 | 300 | |

First Component, Composition 29

| | % | Grams | Phase |
|---|---|---|---|
| Water | 8.3 | 12.45 | A |
| 50% H₂0₂ | 8.4 | 12.6 | A |
| TKPP | 0.2 | 0.3 | A |
| EDTA | 0.5 | 0.75 | |
| KNO₃ | 2 | 3 | |
| Glycerin | 17.55 | 26.325 | A2 |
| carbamide peroxide | 4.5 | 6.75 | A2 |
| Antifoaming agent | QS | QS | A2 |
| Klucel GF | 37 | 55.5 | B |
| Propylene Glycol | 10.05 | 15.075 | B |
| Polawax NF | 6 | 9 | B |
| Combine A & B in “Kitchenaide” | | | C |
| Timica blend | 0.5 | 0.75 | |
| TiO₂ | 0 | 0 | |
| Cabosil EH-5 | 4.5 | 6.75 | C |
| Natural Mint | 0.5 | 0.75 | C |
| | 100 | 150 | |

To summarize, the range of ingredients in the first component in the above examples are as follows:

a 50% hydrogen peroxide solution is, for example, from about 2% to about 15%, more for example, from about 4% to about 12%; carbamide peroxide is, for example, from about 3% to about 20%, more for example, from about 4% to about 17.0%; aerosol, such as Cabosil EH-5, is, for example, from about 2% to about 5%, more for example, about 4.5%; glycerin is, for example, from about 5% to about 25%, more for example, from about 6% to about 20.0%; gelling agent such as Klucel GF is, fro example, from about 1% to about 40%, more for example, from about 2% to about 38%, Polawax NF is, for example, from about 5% to about 10%, more for example, from about 5% to about 7%; propylene glycol is, for example, from about 5% to about 15%, more for example, from about 7% to about 13%, and timica blend/titanium dioxide and mica/“Englehard” is, for example, from about 0.2% to about 1%, more for example, from about 0.25% to about 0.75%. Other ranges may be used without detracting from the present invention.

Second Component, Composition 1

| | % | Grams | Phase |
|---|---|---|---|
| Water | 9.35 | 28.05 | A |
| 50% H₂O₂ | 8 | 24 | A |
| 10% KOH | QS | QS | A |
| Ca(NO₃)₂ | 0.5 | 1.5 | A |
| KNO₃ | 0 | 0 | A |
| Glycerin | 21 | 63 | A2 |
| carbamide peroxide | 5.1 | 15.3 | A2 |
| Antifoaming Agent | QS | QS | A2 |
| Klucel GF | 40 | 120 | B |
| Propylene Glycol | 8.3 | 24.9 | B |
| Polawax NF | 1.25 | 3.75 | B |
| Combine A & B in “:Kitchenaide” | | | C |
| Cabosil EH-5 | 6 | 18 | C |
| Natural Mint | 0.5 | 1.5 | C |
| | 100 | 300 | |

Second Component, Composition 2

| | % | Grams | Phase |
|---|---|---|---|
| Water | 4.72 | 14.46 | A |
| 10% KOH | QS | QS | A |
| Ca(NO₃)₂ | 0.5 | 1.5 | A |
| Glycerin | 20 | 60 | A2 |
| carbamide peroxide | 16 | 48 | A2 |
| Antifoaming Agent | QS | QS | A2 |
| Klucel GF | 35 | 105 | B |
| Propylene Glycol | 12.18 | 36.54 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | 300 | |

Second Component, Composition 3

| | % | Grams | Phase |
|---|---|---|---|
| Water | 7.02 | 21.06 | A |
| 50% H₂O₂ | 5.8 | 17.4 | A |
| 10% KOH | QS | QS | A |
| Ca(NO₃)₂ | 0.5 | 1.5 | A |
| Glycerin | 20 | 60 | A2 |
| carbamide peroxide | 8 | 24 | A2 |
| Antifoaming Agent | QS | QS | A2 |
| Klucel GF | 35 | 105 | B |
| Propylene Glycol | 12.18 | 36.54 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | 300 | |

Second Component, Composition 4

| | % | Grams | Phase |
|---|---|---|---|
| Water | 9 | 27 | A |
| 50% H₂O₂ | 5.8 | 17.4 | A |
| 10% KOH | QS | QS | A |
| Ca(NO₃)₂ | 0.5 | 1.5 | A |
| Glycerin | 18.7 | 56.1 | A2 |
| carbamide peroxide | 8 | 24 | A2 |
| Antifoaming agent | QS | QS | A2 |
| Klucel GF | 34.5 | 103.5 | B |
| Propylene Glycol | 12 | 36 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | 300 | |

Second Component, Composition 5

| | % | Grams | Phase |
|---|---|---|---|
| Water | 12.5 | 37.5 | A |
| 50% H₂O₂ | 4.2 | 12.6 | A |
| 10% KOH | QS | QS | A |
| Ca(NO₃)₂ | 0.5 | 1.5 | A |
| Glycerin | 16.5 | 49.5 | A2 |
| carbamide peroxide | 6 | 18 | A2 |
| Antifoaming agent | QS | QS | A2 |
| Klucel GF | 37 | 111 | B |
| Propylene Glycol | 11.8 | 35.4 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | 300 | |

Second Component, Composition 6

| | % | Grams | Phase |
|---|---|---|---|
| Water | 12.5 | 37.5 | A |
| 50% H₂O₂ | 4.2 | 12.6 | A |
| 10% KOH | QS | QS | A |
| Ca(NO₃)₂ | 0.5 | 1.5 | A |
| Glycerin | 17 | 51 | A2 |
| carbamide peroxide | 6 | 18 | A2 |
| Antifoaming agent | QS | QS | A2 |
| Klucel GF | 41 | 123 | B |
| Propylene Glycol | 12.3 | 36.9 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 7

| | % | Grams | Phase |
|---|---|---|---|
| Water | 13.5 | 40.5 | A |
| 50% H₂O₂ | 4.2 | 12.6 | A |
| 10% KOH | QS | QS | A |
| Ca(NO₃)₂ | 0.5 | 1.5 | A |
| KNO₃ | 2 | 6 | A |
| Glycerin | 14.5 | 43.5 | A2 |
| carbamide peroxide | 6 | 18 | A2 |
| Antifoaming agent | QS | QS | A2 |
| Klucel GF | 41 | 123 | B |
| Propylene Glycol | 11.8 | 35.4 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 8

| | % | Grams | Phase |
|---|---|---|---|
| Water | 12.5 | 37.5 | A |
| 50% H₂O₂ | 4.2 | 12.6 | A |
| 10% KOH | QS | QS | A |
| Ca(NO₃)₂ | 0.5 | 1.5 | A |
| KNO₃ | 2 | 6 | A |
| Glycerin | 15 | 45 | A2 |
| carbamide peroxide | 6 | 18 | A2 |
| Antifoaming agent | QS | QS | A2 |
| Klucel GF | 39 | 117 | B |
| Propylene Glycol | 12.3 | 36.9 | B |
| Polawax NF | 2 | 6 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 9

| | % | Grams | Phase |
|---|---|---|---|
| Water | 13.25 | 39.75 | A |
| 50% H₂O₂ | 4.2 | 12.6 | A |
| 10% KOH | QS | QS | A |
| Ca(NO₃)₂ | 0.5 | 1.5 | A |
| KNO₃ | 2 | 6 | A |
| Glycerin | 19 | 57 | A2 |
| carbamide peroxide | 6 | 18 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 10

| | % | Grams | Phase |
|---|---|---|---|
| Water | 13.75 | 41.25 | A |
| 50% H₂O₂ | 4.2 | 12.6 | A |
| 10% KOH | QS | QS | A |
| KNO₃ | 2 | 6 | A |
| Glycerin | 19 | 57 | A2 |
| carbamide peroxide | 6 | 18 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 11

| | % | Grams | Phase |
|---|---|---|---|
| Water | 13.45 | 40.35 | A |
| 50% H₂O₂ | 4.2 | 12.6 | A |
| 10% KOH | QS | QS | A |
| Ca(NO₃)₂ | 0.5 | 1.5 | A |
| Na Edatate | 0.3 | 0.9 | A |
| KNO₃ | 2 | 6 | A |
| Glycerin | 18.5 | 55.5 | A2 |
| carbamide peroxide | 6 | 18 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 12

| | % | Grams | Phase |
|---|---|---|---|
| Water | 12.95 | 38.85 | A |
| 50% H₂O₂ | 4.2 | 12.6 | A |
| 10% KOH | QS | QS | A |
| Ca(NO₃)₂ | 0.5 | 1.5 | A |
| Na Edatate | 0.3 | 0.9 | A |
| KNO₃ | 2 | 6 | A |
| Glycerin | 19 | 57 | A2 |
| carbamide peroxide | 6 | 18 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 13

| | % | Grams | Phase |
|---|---|---|---|
| Water | 13.25 | 39.75 | A |
| 50% H₂O₂ | 4.2 | 12.6 | A |
| 10% KOH | QS | QS | A |
| Ca(NO₃)₂ | 0.5 | 1.5 | A |
| KNO₂ | 2 | 6 | A |
| Glycerin | 19 | 57 | A2 |
| carbamide peroxide | 6 | 18 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| 50% KOH | QS | QS | C |
| | 100 | 300 | |

Second Component, Composition 14

| | % | Grams | Phase |
|---|---|---|---|
| Water | 15.05 | 45.15 | A |
| 50% H₂O₂ | 8.4 | 25.2 | A |
| 10% KOH | QS | QS | A |
| Ca(NO₃)₂ | 0.5 | 1.5 | A |
| KNO₃ | 2 | 6 | A |
| Glycerin | 19 | 57 | A2 |
| carbamide peroxide | 0 | 0 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| 50% KOH | QS | QS | C |
| | 100 | 300 | |

Second Component, Composition 15

| | % | Grams | Phase |
|---|---|---|---|
| Water | 10.25 | 30.75 | A |
| 50% H₂O₂ | 10.1 | 30.3 | A |
| 10% KOH | QS | | A |
| Ca(NO₃)₂ | 0.5 | 1.5 | |
| KNO₃ | 2 | 6 | |
| Glycerin | 14 | 42 | A2 |
| carbamide peroxide | 8.1 | 24.3 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 16

| | % | Grams | Phase |
|---|---|---|---|
| Water | 10.25 | 30.75 | A |
| 50% H₂O₂ | 10.1 | 30.3 | A |
| 10% KOH | QS | | A |
| Ca(NO₃)₂ | 0.5 | 1.5 | |
| KNO₃ | 2 | 6 | |
| Glycerin | 14 | 42 | A2 |
| carbamide peroxide | 8.1 | 24.3 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 17

| | % | Grams | Phase |
|---|---|---|---|
| Water | 13.75 | 41.25 | A |
| 50% H₂O₂ | 3.6 | 10.8 | A |
| 10% KOH | QS | QS | A |
| Ca(NO₃)₂ | 0.5 | 1.5 | A |
| KNO₃ | 2 | 6 | A |
| Glycerin | 20 | 60 | A2 |
| carbamide peroxide | 5.1 | 15.3 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 18

| | % | Grams | Phase |
|---|---|---|---|
| Water | 13.5 | 40.5 | A |
| 50% H₂O₂ | 3.85 | 11.55 | A |
| 10% KOH | QS | QS | A |
| Ca(NO₃)₂ | 0.5 | 1.5 | A |
| KNO₃ | 2 | 6 | A |
| Glycerin | 19.6 | 58.8 | A2 |
| carbamide peroxide | 5.5 | 16.5 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 19

| | % | Grams | Phase |
|---|---|---|---|
| Water | 10.25 | 30.75 | A |
| 50% H₂O₂ | 5.3 | 15.9 | A |
| 10% KOH | QS | | A |
| Ca(NO₃)₂ | 0.5 | 1.5 | |
| KNO₃ | 2 | 6 | |
| Glycerin | 12.3 | 36.9 | A2 |
| carbamide peroxide | 14.6 | 43.8 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 20

| | % | Grams | Phase |
|---|---|---|---|
| Water | 5 | 15 | A |
| 50% H₂O₂ | 11.8 | 35.4 | A |
| 10% KOH | QS | | A |
| Ca(NO₃)₂ | 0.5 | 1.5 | |
| KNO₃ | 2 | 6 | |
| Glycerin | 9.45 | 28.35 | A2 |
| carbamide peroxide | 16.2 | 48.6 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 21

| | % | Grams | Phase |
|---|---|---|---|
| Water | 13.5 | 40.5 | A |
| 50% H₂O₂ | 3.85 | 11.55 | A |
| 10% KOH | QS | QS | A |
| Ca(NO₃)₂ | 0.5 | 1.5 | A |
| KNO₃ | 2 | 6 | A |
| Glycerin | 19.6 | 58.8 | A2 |
| carbamide peroxide | 5.5 | 16.5 | A2 |
| Antifoaming agent | QS | QS | A2 |
| Klucel GF | 39 | 117 | B |
| Propylene Glycol | 8.3 | 24.9 | B |
| Crodaphos | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 22

| | % | Grams | Phase |
|---|---|---|---|
| Water | 10.25 | 30.75 | A |
| 50% H₂O₂ | 10.1 | 30.3 | A |
| 10% KOH | QS | | A |
| Ca(NO₃)₂ | 0.5 | 1.5 | |
| KNO₃ | 2 | 6 | |
| Glycerin | 14 | 42 | A2 |
| carbamide peroxide | 8.1 | 24.3 | A2 |
| Antifoaming agent | QS | QS | A2 |
| Klucel GF | 39 | 117 | B |
| Propylene Glycol | 8.3 | 24.9 | B |
| Crodaphos | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 23

| | % | Grams | Phase |
|---|---|---|---|
| Water | 13.25 | 39.75 | A |
| 50% H₂O₂ | 3.3 | 9.9 | A |
| 10% KOH | QS | QS | A |
| Ca(NO₃)₂ | 0.5 | 1.5 | A |
| KNO₃ | 2 | 6 | A |
| Glycerin | 20.4 | 61.2 | A2 |
| carbamide peroxide | 5.5 | 16.5 | A2 |
| Antifoaming agent | QS | QS | A2 |
| Klucel GF | 39 | 117 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 24

| | % | Grams | Phase |
|---|---|---|---|
| Water | 13.25 | 39.75 | A |
| 50% H₂O₂ | 3.3 | 9.9 | A |
| 10% KOH | QS | QS | A |
| Ca(NO₃)₂ | 0.5 | 1.5 | A |
| KNO₃ | 0 | 0 | A |
| Glycerin | 21.4 | 64.2 | A2 |
| carbamide peroxide | 5.5 | 16.5 | A2 |
| Antifoaming agent | QS | QS | A2 |
| Klucel GF | 40 | 120 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 25

| | % | Grams | Phase |
|---|---|---|---|
| Water | 13.75 | 41.25 | A |
| 50% H₂O₂ | 3.6 | 10.8 | A |
| 10% KOH | QS | QS | A |
| Ca(NO₃)₂ | 0.5 | 1.5 | A |
| KNO₃ | 0 | 0 | A |
| Glycerin | 21 | 63 | A2 |
| Carbamide peroxide | 5.1 | 15.3 | A2 |
| Antifoaming agent | QS | QS | A2 |
| Klucel GF | 40 | 120 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 26

| | % | Grams | Phase |
|---|---|---|---|
| Water | 10.25 | 30.75 | A |
| 50% H₂O₂ | 10.1 | 30.3 | A |
| 10% KOH | QS | | A |
| Ca(NO₃)₂ | 0.5 | 1.5 | |
| KNO₃ | 0 | 0 | |
| Glycerin | 15 | 45 | A2 |
| carbamide peroxide | 8.1 | 24.3 | A2 |
| Antifoaming agent | QS | QS | A2 |
| Klucel GF | 40 | 120 | B |
| Propylene Glycol | 8.3 | 24.9 | B |
| Polawax NF | 1.25 | 3.75 | B |
| Combine A & B in “Kitchenaide” | | | C |
| Cabosil EH-5 | 6 | 18 | C |
| Natural Mint | 0.5 | 1.5 | C |
| | 100 | 300 | |

Second Component, Composition 27

| | % | Grams | Phase |
|---|---|---|---|
| 50% Hydrogen peroxide | 3.62 | 10.86 | A |
| 50% KOH | QS | QS | A |
| CaCO₃ | 0.2 | 0.6 | A |
| Glycerin | 20 | 60 | A2 |
| carbamide peroxide | 17 | 51 | A2 |
| Antifoaming agent | QS | QS | A2 |
| Klucel GF | 35 | 105 | B |
| Propylene Glycol | 12.68 | 38.04 | B |
| Polawax NF | 6.5 | 19.5 | B |
| Combine A & B in “Kitchenaide” | | | C |
| Cabosil EH-5 | 4.5 | 13.5 | C |
| Natural Mint | 0.5 | 1.5 | C |
| | 100 | 300 | |

To summarize, the range of ingredients in the second component in the above examples are as follows:

a 50% hydrogen peroxide solution is, fro example, from about 2% to about 15%, more for example, from about 4% to about 12%; carbamide peroxide is, for example, from about 3% to about 15%, more for example, from about 5% to about 12.0%; aerosol, such as Cabosil EH-5, is, for example, from about 2% to about 10%, more for example, about 4% to about 6%, and lower ranges are present for foamable or foamed compositions; glycerin is, for example, from about 5% to about 25%, more for example, from about 9% to about 22%; gelling agent such as Klucel GF is, for example, from about 25% to about 50%, more for example, from about 30% to about 45%, Polawax NF is, for example, from about 1% to about 10%, more for example, from about 1% to about 7%; and propylene glycol is, for example, from about 5% to about 15%, more for example, from about 7% to about 13%. Other ranges may be sued without detracting from the present invention.

The following one-component composition was made as follows:

Composition 1

| | % | Grams | Phase |
|---|---|---|---|
| Propylene Glycol | 34.9 | 104.7 | A |
| Klucel GF | 2.09 | 6.27 | A |
| Peppermint PE-05523 | 0.5 | 1.5 | B |
| Calcium Phosphate, tribasic | 0.5 | 1.5 | B |
| Cab-O-Sil EH-5 | 4.5 | 13.5 | B |
| Carbamide Peroxide | 4.5 | 13.5 | B |
| Propylene Glycol | 9.835 | 29.505 | C |
| Glycerin | 17.675 | 53.025 | C |
| Crodaphos | 6 | 18 | C |
| Purified Water | 8.3 | 24.9 | D |
| Hydrogen Peroxide 50% | 8.4 | 25.2 | D |
| NaH2PO4 | 0.4 | 1.2 | D |
| Na2HPO4 | 0.4 | 1.2 | D |
| Potassium Nitrate | 2 | 6 | D |
| H3PO4 | QS | QS | D |
| KOH 50% solution | QS | QS | D |
| | 100 | 300 | |

The ingredients in phase A were premixed together, after which the ingredients in B were added to the premix and warmed to 37 degrees C. The ingredients in C were heated to 93-105 degrees C. to melt the components and blended. The mixture of A and B was heated to 55 degrees C. before adding to C. The mixture of ABC was blended until homogeneous. The ingredients in D were mixed separately until the components dissolved. The pH was adjusted with H3PO4 and KOH. The ingredients in D were then mixed with ABC and blended until homogeneous.

The foam made with any of the above compositions are very long lasting, for example, more than 8 hours, when the amounts of gelling agents and similar ingredients remained the same as shown in the above examples. For some of the compositions of the second component, the foam formed was not as stiff as the foam formed with some of the first component compositions due to the lower viscosities of these second components.

Having described the invention with reference to accompanying illustrations and examples of the invention, it is contemplated that other changes can be made without departing from the spirit or scope of the invention as set forth in the appended claims.

1. A whitening composition comprising at least one source of peroxide and at least one source of calcium salt, wherein said salt provides sustained release of calcium and phosphorous ions in the presence of water for forming hydroxyapatite.

2. The whitening composition of claim 1 wherein said salt comprises a calcium salt of glycerophosphate, lactate, gluconate, fumarate, acetate, chloride or mixtures thereof; α-tricalcium phosphate, β-tricalcium phosphate, tetracalcium phosphate, monocalcium phosphate monobasic, dicalcium phosphate anhydrous, dicalcium phosphate dihydrate, octacalcium phosphate or mixtures thereof.

3. The whitening composition of claim 1 wherein said composition is unfoamed, foamed, foamable or comprises at least one foaming agent.

4. The whitening composition of claim 1 wherein said composition is packaged in a container, contained in a carrier, coated on a substrate or combinations thereof.

5. The whitening composition of claim 1 wherein said salt is in a particulate form having a particle size of less than about 50 um.

6. The whitening composition of claim 3 wherein said foamed or foamable composition has a half life of at least about 1 hour.

7. The whitening composition of claim 4 wherein said container comprises a one compartment container, a two barrel syringe or a multiple compartment container.

8. The whitening composition of claim 4 wherein said container comprises a dispensing pump, a dispensing tip, a dispensing tip adapted for foaming, a dispensing tip including a mixer or combinations thereof.

9. The whitening composition of claim 4 wherein said carrier or substrate may include a substantially insoluble film, a substantially soluble substrate or an erodible film.

10. The whitening composition of claim 9 wherein said carrier or substrate is adapted for affixing onto at least one tooth by means of an adhesive.

11. The whitening composition of claim 1 wherein said composition is a one-component composition comprising at least one source of peroxide, at least one source of soluble or sparingly soluble calcium phosphate salt, and at least one gelling agent, wherein said salt is capable of sustained release of both calcium and phosphate ions into saliva upon exposure of the particle thereto to form a hydroxyapatite.

12. The whitening composition of claim 1 wherein said composition is a 2-component composition having a first component comprising at least one source of peroxide, and at least one gelling agent; and a second component comprising at least one source of soluble or sparingly soluble calcium, strontium and/or mixtures thereof in particulate form, and at least one gelling agent, wherein said source of calcium provides sustained release of calcium ions in the presence of saliva or water for forming hydroxyapatite upon combination with a source of phosphate already present in the saliva, or on the tooth or teeth.

13. The composition of claim 1 wherein said composition further comprises at least one source of phosphate.

14. The composition of claim 12 wherein said composition further comprises an amorphous calcium or strontium compound.

15. The composition of claim 1 further comprising a de-sensitizing agent.

16. The composition of claim 1 further comprising an ingredient an ingredient selected from a group consisting of a de-sensitizing agent, an antibacterial agent, a fluoridating agent, a vitamin supplement, an anti-staining agent, an anti-plaque agent and mixtures thereof.

17. A foamed 2-component composition having a first component comprising at least one source of peroxide, and at least one gelling agent; and a second component comprising at least one source of calcium, strontium and/or mixtures thereof, wherein said source of calcium provides sustained release of calcium ions in the presence of water or saliva, for forming hydroxyapatite upon combination with a source of phosphate already present in the saliva orion the tooth or teeth.

18. The composition of claim 17 wherein said composition further comprises an amorphous calcium or strontium compound.

19. The composition of claim 17 further comprising at least one source of phosphate.

20. The composition of claim 17 The whitening composition of claim 1 wherein said salt comprises a calcium salt of glycerophosphate, lactate, gluconate, fumarate, acetate, chloride or mixtures thereof; α-tricalcium phosphate, β-tricalcium phosphate, tetracalcium phosphate, monocalcium phosphate monobasic, dicalcium phosphate anhydrous, dicalcium phosphate dihydrate, octacalcium phosphate or mixtures thereof.

21. The composition of claim 17 further comprising an ingredient selected from a group consisting of a de-sensitizing agent, an antibacterial agent, a fluoridating agent, a vitamin supplement, an anti-staining agent, an anti-plaque agent and mixtures thereof.

22. A composite comprising:
a substrate; and coated on at least one side of the substrate is a whitening composition comprising at least one source of peroxide, and at least one sparingly soluble or soluble calcium salt in dry particle form;
wherein said salt is capable of sustained release of both calcium and phosphate ions into saliva upon exposure of the particle thereto to form hydroxyapatite.

23. The composite of claim 22 wherein said salt comprises a calcium salt of glycerophosphate, lactate, gluconate, fumarate, acetate, chloride or mixtures thereof; α-tricalcium phosphate; β-tricalcium phosphate; tetracalcium phosphate; monocalcium phosphate monobasic; dicalcium phosphate anhydrous; dicalcium phosphate dehydrate; octacalcium phosphate; or mixtures thereof.

24. The composite of claim 22 further comprising a source of phosphate in particulate form.

25. The composite of claim 24 wherein said source of phosphate comprises monosodium phosphate (NaH₂PO₄), disodium phosphate, tetrapotassium pyrophosphate or mixtures thereof.

26. The composite of claim 22 wherein said composition comprises a low peroxide content.

27. The composite of claim 26 wherein said low peroxide content whitening composition further comprises an ingredient selected from a group consisting of a de-sensitizing agent, an antibacterial agent, a fluoridating agent, a vitamin supplement, an anti-staining agent, an anti-plaque agent and mixtures thereof.

28. The composite of claim 22 wherein said substrate comprises a substantially water insoluble, a substantially water soluble, or a water erodible layer.

29. The composite of claim 28 wherein said soluble layer comprises hydroxyethyl cellulose, hydroxypropyl cellulose, hydroxyethylmethyl cellulose, hydroxypropylmethyl cellulose, polyvinyl alcohol, polyethylene glycol, polyethylene oxide, or ethylene oxide-propylene oxide co-polymers, other water soluble polymers or combinations thereof.

30. The composite of claim 28 wherein said erodible layer comprises a hydrophilic polymer and a hydrophobic polymer.
